(19) United States
(12) Patent Application Publication
(10) Pub. No.: US 2025/0205474 A1
(43) Pub. Date: Jun. 26, 2025

(54) BLOOD PUMP WITH SENSORS

(71) Applicant: ABIOMED, Inc., Danvers, MA (US)

(72) Inventors: Zhongwei Qi, Danvers, MA (US); Michael Finnegan, Danvers, MA (US); Qingchao Kong, Danvers, MA (US); Cam Loughlin, Danvers, MA (US); Zhenghong Tao, Danvers, MA (US)

(73) Assignee: ABIOMED, Inc., Danvers, MA (US)

(21) Appl. No.: 19/001,895

(22) Filed: Dec. 26, 2024

Related U.S. Application Data
(60) Provisional application No. 63/614,732, filed on Dec. 26, 2023.

(51) Int. Cl. A61M 60/531; A61M 60/13; A61M 60/585

(52) U.S. Cl. CPC A61M 60/531; A61M 60/13; A61M 60/585; A61M 2205/04; A61M 2205/3306; A61M 2205/3327; A61M 2205/3331; A61M 2205/3365; A61M 2205/502; A61M 2210/125; A61M 2230/005; A61M 2230/30

(57) ABSTRACT

Disclosed is a blood pump. The blood pump may include a catheter. The blood pump may include a pump housing operably coupled to a distal end of the catheter. The blood pump may include a first sensor (such as an optical sensor, a pressure sensor, etc.) disposed around the catheter or at a distal end of the catheter. The blood pump may include a second sensor disposed distal to the catheter. The first sensor may be disposed at a distance d from the pump housing, where d may be less than ⅓ a length of the catheter. In operation, a controller may be configured to control a speed of the blood pump based on a value from the first sensor and a value from the second sensor.

### CROSS-REFERENCE TO RELATED APPLICATION

The present application claims priority to U.S. Provisional Pat. App. No. 63/614,732, filed Dec. 26, 2023, the entirety of which is incorporated by reference herein.

### TECHNICAL FIELD

The present disclosure is drawn to blood pumps with sensors, and specifically to blood pumps with sensors at or near a distal end of a catheter.

### BACKGROUND

Intravascular blood pumps are often used to control the flow of blood from one part of a patient's body to another. Blood pumps may include sensors, to allow a care provider to understand what is occurring at the location of the blood pump. Further, some blood pumps may use the sensor readings to automatically control a treatment provided to a patient; for example, a speed of the blood pump may be adjusted based on sensor readings. However, in some cases, those sensors may provide false readings, which may lead to unfavorable treatment decisions.

### BRIEF SUMMARY

In various aspects, a blood pump may be provided. The blood pump may include a catheter. The catheter may include one or more lumens extending from a first end to a second end. The catheter may include one or more walls defining a first lumen extending from a first end to a second end, each wall having a wall thickness. One or more sensor lumens may extend through at least one wall of the one or more walls.

The blood pump may include a pump housing operably coupled to a distal end of the catheter. The pump housing may be an expandable pump housing.

The blood pump may include a first sensor disposed around the catheter or at a distal end of the catheter. The first sensor may be disposed at a distance d from the pump housing. The distance d may be less than ⅓ a length of the catheter. Distance d may be less than or equal to 10% of the length of the catheter. The first sensor may be an optical sensor. The first sensor may be a pressure sensor. The first sensor may be disposed at a coupling between the catheter and the pump housing.

The blood pump may include a second sensor disposed distal to the catheter. The second sensor may be an optical sensor. The second sensor may be a pressure sensor.

The blood pump may include a cannula coupled to a distal end of the pump housing.

The blood pump may include a plurality of optical fibers extending at least partially though the one or more lumens. The blood pump may include one or more optical fibers, each of the one or more optical fibers disposed in at least one of the one or more lumens.

The blood pump may include at least one additional sensor disposed around the catheter. The blood pump may include at least one additional sensor disposed distal to the catheter. The first sensor may be disposed at a coupling between the catheter and the pump housing.

In various aspects, a blood pump system may be provided. The system may include an embodiment of a blood pump as disclosed herein. The system may include a controller operably coupled to the blood pump. The controller may be configured to receive information from the first sensor and the second sensor.

The information from the first sensor may include values representative of a first pressure in a first part of a human body. The information from the second sensor may include values representative of a second pressure in a second part of a human body. The controller may be configured to display the first pressure and/or second pressure. The controller may be configured to calculate a value based on the first pressure and second pressure. The controller may be configured to control the blood pump based on the first pressure and second pressure. The controller may be configured to control the blood pump based on the first pressure and second pressure, and a value received from at least one additional sensor disposed on or around the catheter and/or disposed distal to the catheter.

In various aspects, a method for operating a blood pump may be provided. The method may include receiving a measured first pressure value from a sensor disposed on a catheter and a measured second pressure value from a sensor disposed distal to the catheter. The method may include adjusting a speed of a blood pump motor based on the measured first pressure value and the measured second pressure value.

### BRIEF DESCRIPTION OF DRAWINGS

The accompanying drawings, which are incorporated in and constitute a part of this specification, illustrate embodiments of the present invention and, together with a general description of the invention given above, and the detailed description of the embodiments given below, serve to explain the principles of the present invention.

FIG. 1 is an illustration of a system.

FIGS. 2, 3A and 3B are illustrations of portions of blood pump systems.

FIGS. 4A and 4B are illustrations of a location of first sensors on a blood pump.

FIG. 5 is an illustration of a distal end of a blood pump.

FIG. 6 is an illustration of a catheter.

FIG. 7 is a flowchart of a method.

It should be understood that the appended drawings are not necessarily to scale, presenting a somewhat simplified representation of various features illustrative of the basic principles of the invention. The specific design features of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity or illustration.

### DETAILED DESCRIPTION

The following description and drawings merely illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be only for illustrative purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or, unless otherwise indicated (e.g., “or else” or “or in the alternative”). Also, the various embodiments described herein are not necessarily mutually exclusive, as some embodiments can be combined with one or more other embodiments to form new embodiments.

The numerous innovative teachings of the present application will be described with particular reference to the presently preferred exemplary embodiments. However, it should be understood that this class of embodiments provides only a few examples of the many advantageous uses of the innovative teachings herein. In general, statements made in the specification of the present application do not necessarily limit any of the various claimed inventions. Moreover, some statements may apply to some inventive features but not to others. Those skilled in the art and informed by the teachings herein will realize that the invention is also applicable to various other technical areas or embodiments.

As it is known, axial blood pumps may be inserted percutaneously and provide support to a patient's heart for a period of time. Such blood pumps may be inserted into the left ventricle via the femoral artery. In other aspects, such blood pumps may be inserted into the right ventricle. As it is also known, blood pumps may be equipped with optical sensors. In such instances, optical sensors may provide valuable data to the user while a blood pump is operating.

The inventors have recognized that placement of such optical sensors may be challenging. For example, some existing blood pumps may only have one optical sensor although more optical sensors are desired. In another example, optical sensors may only be positioned on the blood pump in places where they will not be damaged during placement and/or support. Furthermore, there may be limited space available on the blood pump surface and thus fewer options available for optical sensor placement.

Accordingly, the inventors have recognized the benefit of including additional optical sensors on the catheter of the blood pump. The inventors have also recognized the benefit of including a ring on the catheter on which one or more optical sensors may be located. Such optical sensors may be positioned such that they measure data in the patient's aorta (for pumps placed in the left ventricle) and/or the inferior vena cava (for pumps placed in the right ventricle).

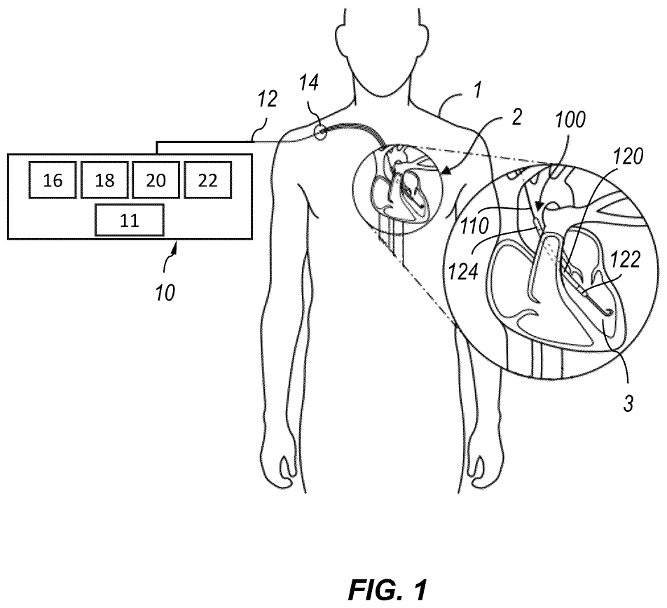

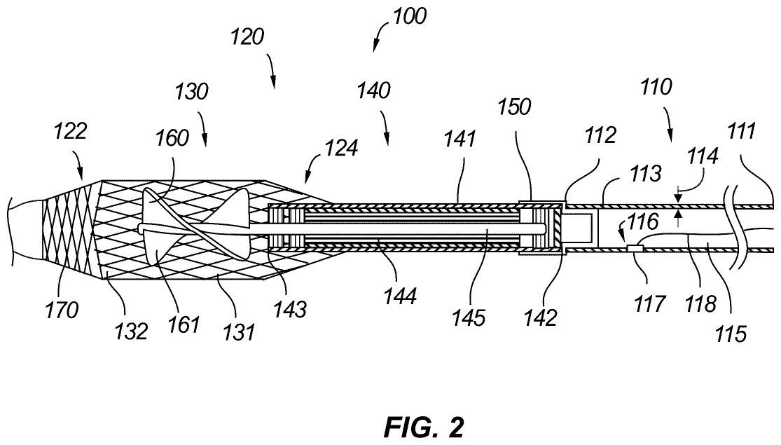

In various aspects, a blood pump system may be provided. Referring to FIG. 1, a blood pump system 100 may be deployed within a blood vessel of a patient 1, such as in a heart 2. The blood pump may include a pump section 120 operably coupled to a distal end of a catheter 110. The blood pump may be configured to cause blood to flow into an inlet 122, through the pump section 120, and out of a blood-flow outlet 124. Here, the blood flow inlet is shown as being disposed within a left ventricle 3, but as will be understood, the pump could be deployed at other locations within a patient. For example, in some embodiments, the blood pump may be deployed in the right ventricle of a patient. The pump may be operably coupled to a controller 10 via, e.g., one or more wires 12 or tubes through an access point 14. In some embodiments, the catheter 110 may house the one or more wires 12. Referring to FIG. 2, a portion of a blood pump system is shown. The blood pump system 100 may include a catheter 110. The catheter may include one or more lumens 115 extending from a first end 111 to a second end 112. The catheter may include one or more walls 113 defining the first lumen 115, each wall having a wall thickness 114. One or more sensor lumens 118 may extend through at least one of the one or more walls 113. One or more optical fibers or electrical connections 116 may be operably coupled to a sensor 117. The sensor and/or the optical fibers or electrical connections operably coupled to the sensor may be disposed at least partially within the sensor lumen 118.

The blood pump may include a pump housing 120 operably coupled to a distal end 112 of the catheter 110. The pump housing may be an expandable pump housing. As shown in FIG. 2, the pump housing may include an expandable portion 130. The pump housing may include a motor portion 140. The motor portion may include a housing 141 having a distal end 143 and a proximal end 142. The proximal end of the motor may be operably coupled to the distal end 112 of the catheter. The motor housing may be disposed around an electric motor 144. The motor may be configured to rotatably drive the impeller. The electric motor may be operably coupled to an impeller 160, e.g., via a drive shaft 145. In such embodiments, the motor may be located outside of the patient and connected to the impeller via a flexible driveshaft extending through the catheter.

In some embodiments, the impeller may be an expandable impeller, and may be disposed within the expandable portion 130 of the pump housing. In other embodiments, the impeller need not be expandable. The impeller may include one or more blades 161 configured to cause blood to flow from the inlet 122 to the outlet 124 when the impeller rotates. The expandable portion may include one or more struts 131. The struts may be a metal or metal alloy. The struts may define one or more apertures 132 between the struts. A mesh filter 170 may be disposed at the inlet 122.

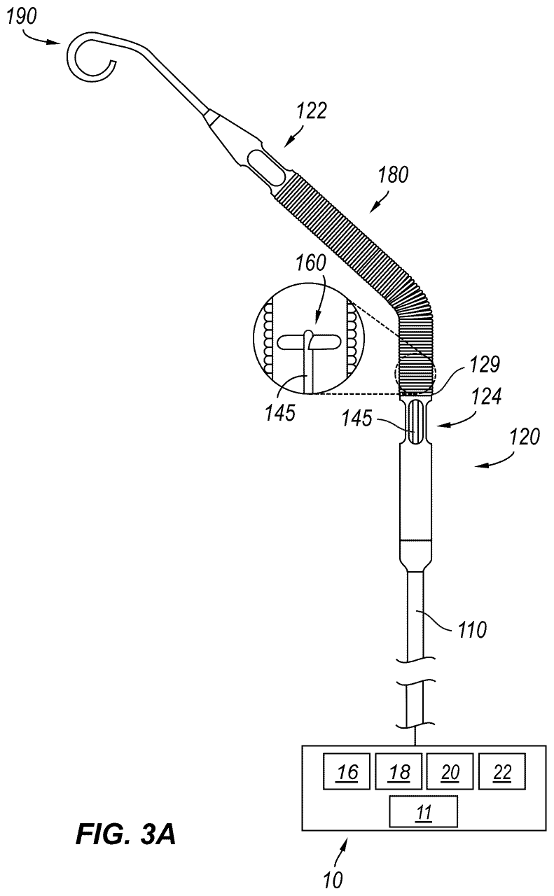

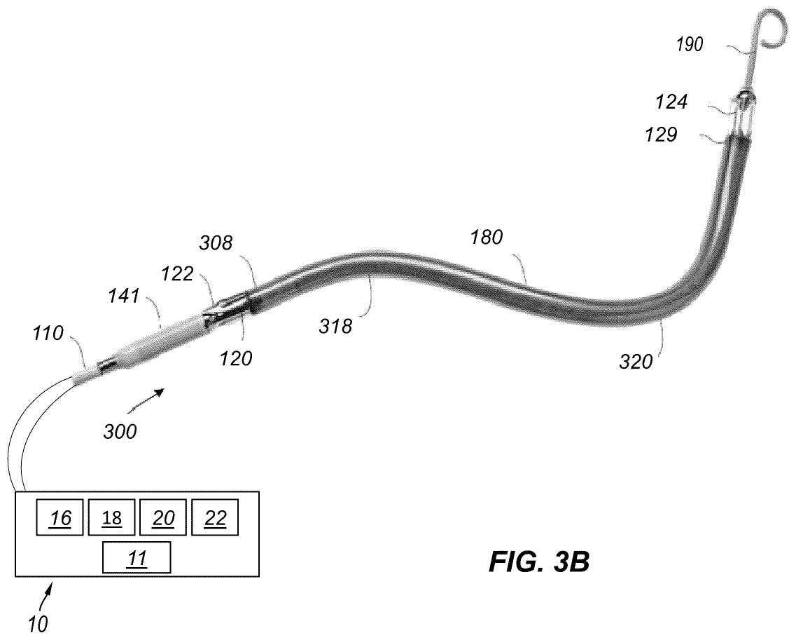

In some embodiments, the pump housing may be directly coupled to the catheter. In some embodiments, a coupling 150 may be disposed between the catheter and the pump housing. A blood pump system with a non-expandable cannula is shown in FIG. 3A. In FIG. 3A, a blood pump system is shown where blood is configured to flow into an inlet 122, through a cannula 180, and out an outlet 124. The pump housing may include the outlet 124. The cannula may be disposed at a distal end 129 of the pump housing. In some embodiments, the cannula may include one or more pre-formed anatomical bends. In some embodiments, the cannula may be flexible. In still some embodiments, the cannula may be capable of straightening (e.g., during insertion over a guidewire) or bending further (e.g., in a patient whose anatomy has tighter dimensions). For example, the cannula may be made of Nitinol. In other embodiments, the cannula may include a shape-memory material configured to allow the cannula to be a different shape (e.g., straight or mostly straight) at room temperatures, and to form bend(s) once the shape-memory material is exposed to the heat of the patient's body. As shown in FIG. 3A, in some embodiments, the impeller may be disposed within outside the pump housing, such as within the cannula. A motor may be located in the pump housing 120. In some embodiments, the motor may be located outside of the patient.

A distal flexible extension 190 may be operably coupled to a distal end of the pump housing. The distal flexible extension may assist with stabilizing and positioning the blood pump in a desired position in the patient's heart. The distal flexible extension may be solid or tubular. In some embodiments, if tubular, the distal flexible extension may be configured to allow a guidewire to be passed through it to further assist in the positioning of the blood pump. As will be appreciated, the distal flexible extension may be of any suitable size. The distal flexible extension may be composed, at least in part, of a flexible material, and may be any suitable shape or configuration. As will also be appreciated, the distal flexible extension may have sections with different stiffnesses and/or different materials. In some embodiments, the blood pump may not include a distal flexible extension.

As described herein, the blood pump may be inserted percutaneously. For example, when used for left heart support, the blood pump may be inserted via a catheterization procedure through the femoral artery or axillary artery, into the aorta, across the aortic valve, and into the left ventricle. Once positioned in this location, the blood pump may deliver blood from the inlet 122, which sits inside the left ventricle, through the cannula 180 and to the outlet 124, which sits in the ascending aorta.

FIG. 3B depicts an exemplary blood pump system 300 adapted for right heart support, in accordance with aspects of the disclosure. As shown in FIG. 3B, a blood pump system adapted for right heart support may include a catheter 110, a motor housing 141, a cannula 180, an inlet 122 arranged at or near the proximal end 308 of the cannula 180, an outlet 124 arranged at or near the distal end 129 of the cannula 180, and an optional distal flexible extension 190 arranged at the distal end of the outlet 124.

As with the exemplary embodiments of FIGS. 1 and 2, a motor may be configured to rotatably drive an impeller (not shown), thereby generating suction sufficient to draw blood into cannula 180 through the inlet 122, and to expel the blood out of cannula 180 through the outlet 124. In that regard, the impeller may be positioned distal of the inlet 122, for example, within the proximal end 308 of the cannula 180 or within a pump housing 120 coupled to the proximal end 308 of the cannula 180. Here as well, in some aspects of the present technology, rather than the impeller being driven by an onboard motor, the impeller may instead be coupled to an elongate drive shaft (or drive cable) which is driven by a motor located external to the patient.

In some embodiment, the cannula 180 of FIG. 3B may serve the same purpose, and may have the same properties and features described above with respect to cannula 180 of FIG. 3A. However, as shown in the exemplary arrangement of FIG. 3B, the cannula 180 may have two pre-formed anatomical bends 318 and 320 based on the portion of the right heart in which it is intended to operate. Here again, despite the existence of bends 318 and 320, the cannula 180 may nevertheless also be flexible, and may thus be capable of straightening (e.g., during insertion over a guidewire), or bending further (e.g., in a patient whose anatomy has tighter dimensions). Further in that regard, cannula 180 may include a shape-memory material configured to allow the cannula 180 to be a different shape (e.g., straight or mostly straight) at room temperatures, and to form bends 318 and/or 320 once the shape-memory material is exposed to the heat of a patient's body. Furthermore, in some embodiments, cannula 180 may also not include bends 318 and/or 320. In such embodiments, cannula 180 may be relatively straight during insertion and capable of bending to accommodate patients' anatomy once the pump is positioned in the patient.

The catheter 110 and optional distal flexible extension 190 of FIG. 3B may serve the same purpose and may have the same properties and features described above with respect to catheter 110 and optional distal flexible extension 190 of FIG. 3A. Likewise, other than being located at opposite ends of the cannula from those of FIG. 3A, the inlet 122 and outlet 124 of FIG. 3B may be similar to the inlet 122 and outlet 124 of FIG. 3A, and thus may have the same properties and features described above. As will be appreciated, although shown with an optional distal flexible extension 190 in FIG. 3B, the pump may not include a distal flexible extension.

Like the exemplary embodiment of FIG. 3A, the intracardiac blood pump assembly 300 of FIG. 3B may also be inserted percutaneously. For example, when used for right heart support, intracardiac blood pump assembly 300 may be inserted via a catheterization procedure through the femoral vein, into the inferior vena cava, through the right atrium, across the tricuspid valve, into the right ventricle, through the pulmonary valve, and into the pulmonary artery. Once positioned in this way, the intracardiac blood pump assembly 300 may deliver blood from the inlet 122, which sits inside the inferior vena cava, through cannula 180, to the outlet 124, which sits inside the pulmonary artery. In some embodiments, the intracardiac blood pump assembly 300 may be inserted via the internal jugular (IJ) vein. For example, in such embodiments, intracardiac blood pump assembly 300 may be inserted via a catheterization procedure through the IJ vein, into the superior vena cava, through the right atrium, across the pulmonary valve, and into the pulmonary artery. Once positioned in this location, the intracardiac blood pump assembly 300 may deliver blood from the inlet 122, which sits inside the superior vena cava, through cannula 180, to the outlet 124, which sits inside the pulmonary artery.

Referring back to FIG. 1, the controller 10 may include one or more memory 16, one or more processors 18, a user interface 20, and one or more sensors, such as current sensors 22. Processor(s) may comprise one or more microcontrollers, one or more microprocessors, one or more application specific integrated circuits (ASICs), one or more digital signal processors, program memory, or other computing components. Processor(s) 18 may be communicatively coupled to the other components (e.g., memory 16, user interface 20, current sensor(s) 22) of control unit 10 and may be configured to control one or more operations of pump 100. As a non-limiting example, control unit 10 may be implemented as an Automated Impella Controller™ from ABIOMED, Inc., Danvers, MA. In some aspects, memory 16 is included as a portion of processor(s) 18 rather than being provided as a separate component.

During operation, processor(s) 18 may be configured to control the electrical power delivered to a motor (e.g., by controlling a power supply (not shown)) by a power supply line (not shown) in wires 12, thereby controlling the speed of the motor. Current sensor(s) 22 may be configured to sense motor current associated with an operating state of the motor, and processor(s) 18 may be configured to receive the output of current sensor(s) 22 as a motor current signal. Processor(s) 18 may further be configured to determine a flow through the pump 100 based, at least in part, on the motor current signal and the motor speed, as described in more detail herein. Current sensor(s) 22 may be included in control unit 10 or may be located along any portion of the power supply line in wires 12. Additionally or alternatively, current sensor(s) 22 may be included in motor 140 and processor(s) 18 may be configured to receive the motor current signal via a data line (not shown) in wires 12 coupled to processor(s) 18 and motor 140.

Memory 16 may be configured to store computer-readable instructions and other information for various functions of the components of control unit 10. In one aspect, memory 16 includes volatile and/or non-volatile memory, such as, an electrically erasable programmable read-only memory (EEPROM).

User interface 20 may be configured to receive user input via one or more buttons, switches, knobs, etc. Additionally, user interface 20 may include a display configured to display information and one or more indicators, such as light indicators, audio indicators, etc., for conveying information and/or providing alerts regarding the operation of pump 100.

The controller 10 of FIG. 3A and FIG. 3B may serve the same purpose, and may have the same properties and features described above with respect to controller 10 of FIG. 1.

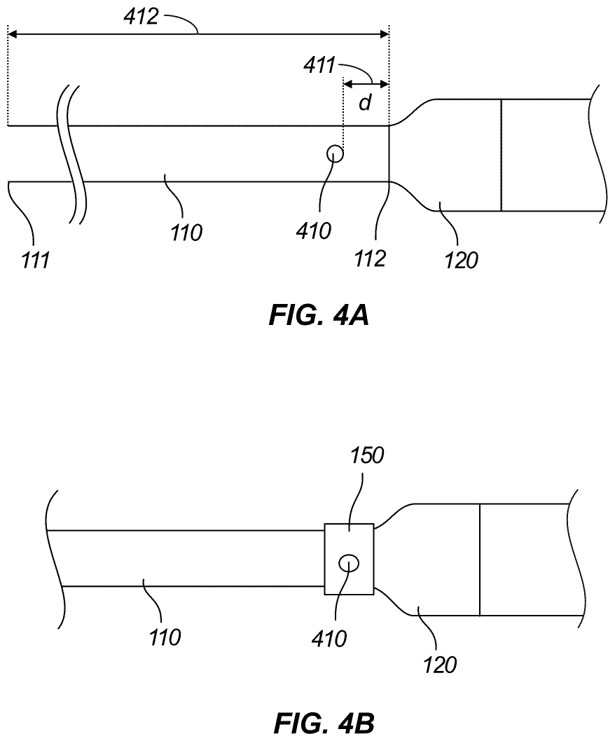

Referring to FIG. 4A, the blood pump may include a first sensor 410 disposed around the catheter or at a distal end 112 of the catheter. The first sensor may be an optical sensor. The first sensor may be a pressure sensor.

The first sensor may be disposed at a distance (“d”) 411. from the pump housing. The distance d may be less than ⅓ a length 412 of the catheter. Distance d may be less than or equal to 20% of the length of the catheter. Distance d may be less than or equal to 10% of the length of the catheter. Distance d may be less than or equal to 8% of the length of the catheter. Distance d may be less than or equal to 6% of the length of the catheter. Distance d may be less than or equal to 5% of the length of the catheter. Distance d may be less than or equal to 4% of the length of the catheter. Distance d may be less than or equal to 3% of the length of the catheter. Distance d may be less than or equal to 2% of the length of the catheter. Distance d may be less than or equal to 1% of the length of the catheter.

Referring to FIG. 4B, the first sensor may be disposed at or on a coupling 150 between the catheter 110 and the pump housing 120.

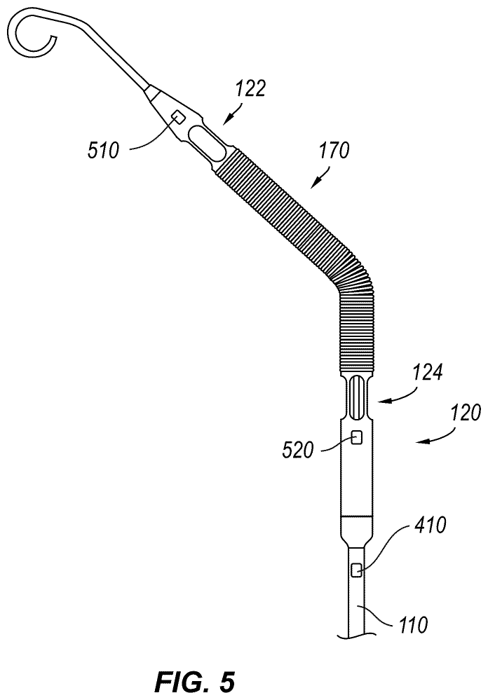

Referring to FIG. 5, the blood pump may include a second sensor 510 disposed distal to the catheter. For example, the second sensor 510 may be disposed on the inlet, as shown in FIG. 5. In another embodiment, the second sensor may be disposed on the outlet of a right-sided pump, such as the outlet in FIG. 3. The second sensor may be an optical sensor. The second sensor may be a pressure sensor.

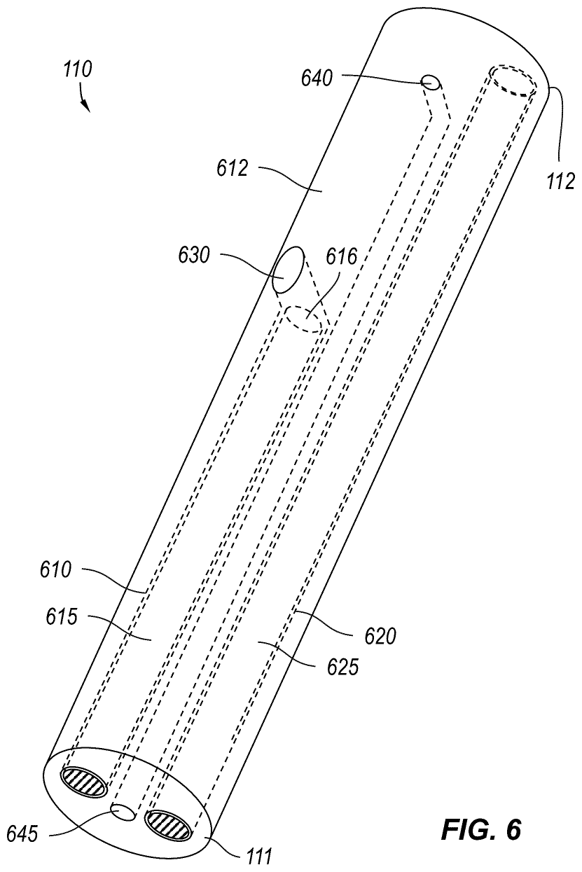

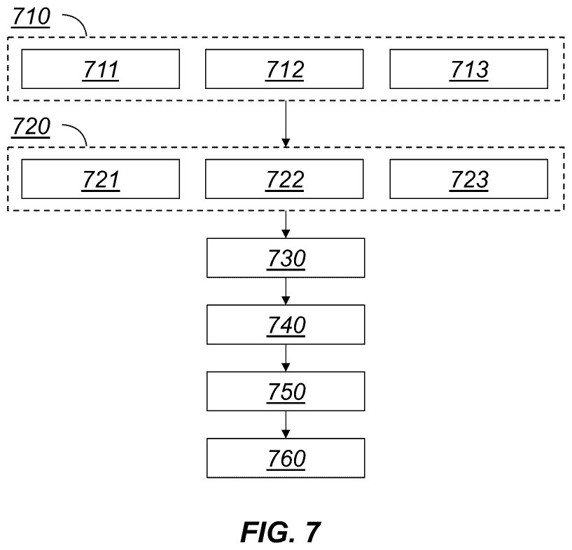

Referring to FIG. 6, the catheter 110 may include a plurality of lumen (including, e.g., first lumen 610 and second lumen 620). The blood pump may include a lumen that extends a distance less than the total length of the catheter. For example, first lumen 610 is shown as beginning at a proximal end 111 of the catheter and ending at an external surface 612 of the catheter, proximal to the distal end 112 of the catheter. The second lumen 620 is shown as beginning at the proximal end and extending to the distal end.

The blood pump may include a plurality of optical fibers (including, e.g., first optical fiber 615 and second optical fiber 625) extending at least partially though the one or more lumens. In some embodiments, two or more fibers extend through a single optical fiber lumen. In some embodiments, a single fiber extends through each optical fiber lumen. In some embodiments, the blood pump may include one or more optical fibers, each of the one or more optical fibers disposed in at least one of the one or more lumens. As seen, in some embodiments, an optical fiber (e.g., first fiber 615) may not extend entirely through a lumen. As shown, an end 616 of the first optical fiber is configured to end proximal the end of the first lumen 610. As shown, a sensor 630 may be coupled to the end 616 of the optical fiber. In some embodiments, the catheter may include a lumen configured to be a purge fluid conduit. In other embodiments, the catheter may include a lumen configured to receive a guidewire.

The blood pump may include at least one additional sensor 640. The additional sensor may be operably coupled to one or more additional lumen 645. As shown in FIG. 6, one or more additional sensor(s) 640 may be disposed around the catheter. As will be appreciated, in FIG. 6, only three lumen are shown, for clarity. It will be understood that more (or less) lumen may be present in other embodiments.

Referring to FIG. 5, the blood pump may include at least one additional sensor 520 disposed distal to the catheter. In FIG. 5, the sensor is shown disposed on an external surface of the pump housing 120, but as will be understood, additional sensors may readily be placed in various locations distal to the catheter, including at or near the inlet 122, at or near the outlet 124, internal to the cannula 180, and/or at or near the distal flexible extension 190.

In various aspects, a blood pump system may be provided. As seen in FIG. 1, the system may include an embodiment of a blood pump 100 as disclosed herein. The system may include a controller 10 operably coupled to the blood pump.

The controller may be configured to perform specific steps of a method.

The method may include receiving 710 information from the sensors. This may include receiving 711 information from the first sensor and receiving information 712 from the second sensor. This may include receiving 713 information from at least one additional sensor (such as sensor 520 in FIG. 5). As will be understood, the information may be received simultaneously (e.g., in parallel), or may be received at different times (e.g., serially) in any order. The information from the first sensor may include values representative of a first pressure in a first part of a human body. The information from the second sensor may include values representative of a second pressure in a second part of a human body.

The controller may be configured to determine 720 a pressure. This may include determining 721 a first pressure (e.g., from information received from the first sensor) and/or determining 722 a second pressure (e.g., from information received from the second sensor). This may include determining 723 a third pressure (e.g., from information received from an additional sensor). The pressure may be determined based on a single reading (e.g., a single piece of information from a sensor). The pressure may be determined based on multiple sensor readings over a period of time. For example, the controller may determine a pressure every two seconds based on sensor information collected over the previous two seconds, such as an average, maximum, or minimum value over the previous two seconds.

The controller may be configured to display 730 the first pressure and/or second pressure. (e.g., on display 11 of the controller 10 in FIG. 1).

The controller may be configured to calculate 740 a value based on the first pressure and second pressure. For example, the controller may be configured to calculate a pressure different between the first pressure (which may be, e.g., a blood pressure within the aorta) and the second pressure (which may be a blood pressure within the left ventricle).

The controller may be configured to control 750 the blood pump based on the first pressure and second pressure. For example, the electrical current required to maintain a desired flow rate of blood from the inlet to the outlet may be based on the first pressure and the second pressure, where a change in electrical current adjusts the speed of the blood pump motor. The controller may be configured to control 760 the blood pump based on the first pressure and second pressure, and a value received from at least one additional sensor disposed on or around the catheter and/or disposed distal to the catheter.

Various modifications may be made to the systems, methods, apparatus, mechanisms, techniques, and portions thereof described herein with respect to the various figures, such modifications being contemplated as being within the scope of the invention. For example, while a specific order of steps or arrangement of functional elements is presented in the various embodiments described herein, various other orders/arrangements of steps or functional elements may be utilized within the context of the various embodiments. Further, while modifications to embodiments may be discussed individually, various embodiments may use multiple modifications contemporaneously or in sequence, compound modifications and the like.

Although various embodiments which incorporate the teachings of the present invention have been shown and described in detail herein, those skilled in the art can readily devise many other varied embodiments that still incorporate these teachings. Thus, while the foregoing is directed to various embodiments of the present invention, other and further embodiments of the invention may be devised without departing from the basic scope thereof. As such, the appropriate scope of the invention is to be determined according to the claims.

1. A blood pump, comprising:
a catheter;
a pump housing operably coupled to a distal end of the catheter;
a first sensor disposed around the catheter or at a distal end of the catheter; and
a second sensor disposed distal to the catheter.

2. (canceled)

3. The blood pump of claim 2, wherein d≤10% of the length of the catheter.

4. The blood pump of claim 1, where in the first sensor is an optical sensor.

5. The blood pump of claim 1, wherein the first sensor is a pressure sensor.

6. The blood pump of claim 1, where in the second sensor is an optical sensor.

7. The blood pump of claim 1, wherein the second sensor is a pressure sensor.

8. The blood pump of claim 1, wherein the pump housing is an expandable pump housing.

9. The blood pump of claim 1, further comprising a cannula coupled to a distal end of the pump housing.

10. The blood pump of claim 1, wherein the catheter comprises one or more lumens extending from a first end to a second end.

11. The blood pump of claim 10, further comprising a plurality of optical fibers extending at least partially though the one or more lumens.

12. The blood pump of claim 10, further comprising one or more optical fibers, each of the one or more optical fibers disposed in each of the one or more lumens.

13. The blood pump of claim 1, further comprising at least one additional sensor disposed around the catheter.

14. The blood pump of claim 1, further comprising at least one additional sensor disposed distal to the catheter.

15. The blood pump of claim 1, wherein the first sensor is disposed at a coupling between the catheter and the pump housing.

16. The blood pump of claim 1, wherein the catheter comprises one or more walls defining a first lumen extending from a first end to a second end, each wall having a wall thickness.

17. The blood pump of claim 16, wherein one or more sensor lumens extend through at least one wall of the one or more walls.

18. A blood pump system, comprising:
a blood pump of claim 1; and
a controller operably coupled to the blood pump, the controller configured to receive information from the first sensor and the second sensor.

19. The blood pump system of claim 18, wherein the information from the first sensor includes values representative of a first pressure in a first part of a human body.

20. The blood pump system of claim 19, wherein the information from the second sensor includes values representative of a second pressure in a second part of a human body.

21-24. (canceled)

25. A method for operating a blood pump, comprising:
receiving a measured first pressure value from a sensor disposed on a catheter and a measured second pressure value from a sensor disposed distal to the catheter; and
adjusting a speed of a blood pump motor based on the measured first pressure value and the measured second pressure value.
